(19) United States
(12) Patent Application Publication
(10) Pub. No.: US 2020/0352662 A1
(43) Pub. Date: Nov. 12, 2020

(54) MANIPULATOR FOR ROBOTIC SURGICAL TOOL

(71) Applicant: Medrobotics Corporation, Raynham, MA (US)

(72) Inventors: Anish Mampetta, Waterbeach Cambridgeshire (GB); Ian J. Darisse, Southborough, MA (US)

(21) Appl. No.: 16/960,159

(22) PCT Filed: Jan. 7, 2019
(86) PCT No.: PCT/US19/12481

§ 371 Date: Jul. 6, 2020

Related U.S. Application Data
(60) Provisional application No. 62/614,225, filed on Jan. 5, 2018.
(60) Provisional application No. 62/614,228, filed on Jan. 5, 2018.
(60) Provisional application No. 62/614,240, filed on Jan. 5, 2018.

(51) Int. Cl. A61B 34/30; B25J 5/00; B25J 5/02; A61B 90/50; A61B 90/30; B25J 9/10

(52) U.S. Cl. CPC A61B 34/30; B25J 5/007; B25J 5/02; A61B 2090/306; A61B 90/30; B25J 9/106; A61B 2034/301; A61B 90/50

(57) ABSTRACT

A system for performing a medical procedure on a patient includes an articulating probe assembly and at least one tool. The articulating probe assembly comprises an inner probe comprising multiple articulating inner links, an outer probe surrounding the inner probe and comprising multiple articulating outer links, and at least two working channels that exit a distal portion of the probe assembly. The at least one tool is configured to translate through one of the at least two working channels. A manipulator is provided for controlling the at least one tool.

### RELATED APPLICATIONS

This application claims the benefit of U.S. Provisional Application No. 62/613,899, filed Jan. 5, 2018, the content of which is incorporated herein by reference in its entirety.

This application claims the benefit of U.S. Provisional Application No. 62/614,223, filed Jan. 5, 2018, the content of which is incorporated herein by reference in its entirety.

This application claims the benefit of U.S. Provisional Application No. 62/614,224, filed Jan. 5, 2018, the content of which is incorporated herein by reference in its entirety.

This application claims the benefit of U.S. Provisional Application No. 62/614,228, filed Jan. 5, 2018, the content of which is incorporated herein by reference in its entirety.

This application claims the benefit of U.S. Provisional Application No. 62/614,225, filed Jan. 5, 2018, the content of which is incorporated herein by reference in its entirety.

This application claims the benefit of U.S. Provisional Application No. 62/614,240, filed Jan. 5, 2018, the content of which is incorporated herein by reference in its entirety.

This application claims the benefit of U.S. Provisional Application No. 62/614,235, filed Jan. 5, 2018, the content of which is incorporated herein by reference in its entirety.

This application is related to U.S. Provisional Application No. 61/921,858, filed Dec. 30, 2013, the content of which is incorporated herein by reference in its entirety.

This application is related to PCT Application No PCT/US2014/071400, filed Dec. 19, 2014, PCT Publication No. WO2015/102939, the content of which is incorporated herein by reference in its entirety.

This application is related to U.S. patent application Ser. No. 14/892,750, filed Nov. 20, 2015, U.S. Publication No. 2016/0256226, now U.S. Pat. No. 10,004,568 issued on Jun. 26, 2018, the content of which is incorporated herein by reference in its entirety.

This application is related to U.S. patent application Ser. No. 15/899,826, filed Feb. 20, 2018, U.S. Publication No. 2018/0250095 the content of which is incorporated herein by reference in its entirety.

This application is related to U.S. Provisional Application No. 61/406,032, filed Oct. 22, 2010, the content of which is incorporated herein by reference in its entirety.

This application is related to PCT Application No PCT/US2011/057282, filed Oct. 21, 2011, PCT Publication No. WO2012/054829, the content of which is incorporated herein by reference in its entirety.

This application is related to U.S. patent application Ser. No. 13/880,525, filed Apr. 19, 2013, U.S. Publication No. 2014/0005683, now U.S. Pat. No. 8,992,421, issued on Mar. 31, 2015, the content of which is incorporated herein by reference in its entirety.

This application is related to U.S. patent application Ser. No. 14/587,166, filed Dec. 31, 2014, U.S. Publication No. 2015/0313449, the content of which is incorporated herein by reference in its entirety.

This application is related to U.S. Provisional Application No. 61/492,578, filed Jun. 2, 2011, the content of which is incorporated herein by reference in its entirety.

This application is related to PCT Application No. PCT/US2012/040414, filed Jun. 1, 2012, PCT Publication No. WO2012/167043, the content of which is incorporated herein by reference in its entirety.

This application is related to U.S. patent application Ser. No. 14/119,316, filed Nov. 21, 2013, U.S. Publication No. 2014/0094825, the content of which is incorporated herein by reference in its entirety.

This application is related to U.S. Provisional Application No. 62/504,175, filed May 10, 2017, the content of which is incorporated herein by reference in its entirety.

This application is related to PCT Application No. PCT/US2018/031774, filed May 9, 2018, PCT Publication No. WO2018/0020898, the content of which is incorporated herein by reference in its entirety.

This application is related to U.S. Provisional Application No. 61/412,733, filed Nov. 11, 2010, the content of which is incorporated herein by reference in its entirety.

This application is related to PCT Application No PCT/US2011/060214, filed Nov. 10, 2011, PCT Publication No. WO2012/078309, the content of which is incorporated herein by reference in its entirety.

This application is related to U.S. patent application Ser. No. 13/884,407, filed May 9, 2013, U.S. Publication No. 2014/0012288, now U.S. Pat. No. 9,649,163, issued on May 16, 2017, the content of which is incorporated herein by reference in its entirety.

This application is related to U.S. patent application Ser. No. 15/587,832, filed May 5, 2017, U.S. Publication No. 2018/0021095, the content of which is incorporated herein by reference in its entirety.

This application is related to U.S. Provisional Application No. 61/472,344, filed Apr. 6, 2011, the content of which is incorporated herein by reference in its entirety.

This application is related to PCT Application No. PCT/US2012/032279, filed Apr. 5, 2012, PCT Publication No. WO2012/138834, the content of which is incorporated herein by reference in its entirety.

This application is related to U.S. patent application Ser. No. 14/008,775, filed Sep. 30, 2013, U.S. Publication No. 2014/0046305, now U.S. Pat. No. 9,962,179, issued on May 8, 2018, the content of which is incorporated herein by reference in its entirety.

This application is related to U.S. patent application Ser. No. 14/944,665, filed Nov. 18, 2015, U.S. Publication No.: 2016/0066938, the content of which is incorporated herein by reference in its entirety.

This application is related to U.S. patent application Ser. No. 14/945,685, filed Nov. 19, 2015, U.S. Publication No. 2016/0066939, the content of which is incorporated herein by reference in its entirety.

This application is related to U.S. Provisional Application No. 61/534,032 filed Sep. 13, 2011, the content of which is incorporated herein by reference in its entirety.

This application is related to PCT Application No. PCT/US2012/054802, filed Sep. 12, 2012, PCT Publication No. WO2013/039999, the content of which is incorporated herein by reference in its entirety.

This application is related to U.S. patent application Ser. No. 14/343,915, filed Mar. 10, 2014, U.S. Publication No. 2014/0371764, now U.S. Pat. No. 9,757,856, issued on Sep. 12, 2017, the content of which is incorporated herein by reference in its entirety.

This application is related to U.S. patent application Ser. No. 15/064,043, filed Mar. 8, 2016, U.S. Publication No. 2016/0262840, now U.S. Pat. No. 9,572,628, issued on Feb. 21, 2017, the content of which is incorporated herein by reference in its entirety.

This application is related to U.S. patent application Ser. No. 15/684,268, filed Aug. 23, 2017, U.S. Publication No. 2017/0368681, the content of which is incorporated herein by reference in its entirety.

This application is related to U.S. Provisional Application No. 61/368,257, filed Jul. 28, 2010, the content of which is incorporated herein by reference in its entirety.

This application is related to PCT Application No PCT/US2011/044811, filed Jul. 21, 2011, PCT Publication No. WO2012/015659, the content of which is incorporated herein by reference in its entirety.

This application is related to U.S. patent application Ser. No. 13/812,324, filed Jan. 25, 2013, U.S. Publication No. 2014/0012287, now U.S. Pat. No. 9,901,410, issued on Feb. 27, 2018, the content of which is incorporated herein by reference in its entirety.

This application is related to U.S. patent application Ser. No. 15/874,189, filed Jan. 18, 2018, U.S. Publication No. 2018-0206923 the content of which is incorporated herein by reference in its entirety.

This application is related to U.S. Provisional Application No. 61/578,582, filed Dec. 21, 2011, the content of which is incorporated herein by reference in its entirety.

This application is related to PCT Application No. PCT/US2012/070924, filed Dec. 20, 2012, PCT Publication No. WO2013/096610, the content of which is incorporated herein by reference in its entirety.

This application is related to U.S. patent application Ser. No. 14/364,195, filed Jun. 10, 2014, U.S. Publication No. 2014/0318299, now U.S. Pat. No. 9,364,955 issued on Jun. 14, 2016, the content of which is incorporated herein by reference in its entirety.

This application is related to U.S. patent application Ser. No. 15/180,503, filed Jun. 13, 2016, U.S. Publication No. 2017/0015007, now U.S. Pat. No. 9,821,477, issued on Nov. 21, 2017, the content of which is incorporated herein by reference in its entirety.

This application is related to U.S. patent application Ser. No. 15/786,901, filed Oct. 18, 2017, U.S. Publication No. 2018/0161992, the content of which is incorporated herein by reference in its entirety.

This application is related to U.S. Provisional Application No. 61/681,340, filed Aug. 9, 2012, the content of which is incorporated herein by reference in its entirety.

This application is related to PCT Application No. PCT/US2013/054326, filed Aug. 9, 2013, PCT Publication No. WO2014/026104, the content of which is incorporated herein by reference in its entirety.

This application is related to U.S. patent application Ser. No. 14/418,993, filed Feb. 2, 2015, U.S. Publication No. 2015/0282835, now U.S. Pat. No. 9,675,380 issued on Jun. 13, 2017, the content of which is incorporated herein by reference in its entirety.

This application is related to U.S. patent application Ser. No. 15/619,875, filed Jun. 12, 2017, U.S. Publication No. 2018/0021060, the content of which is incorporated herein by reference in its entirety.

This application is related to U.S. Provisional Application No. 61/751,498, filed Jan. 11, 2013, the content of which is incorporated herein by reference in its entirety.

This application is related to PCT Application No. PCT/US2014/010808, filed Jan. 9, 2014, PCT Publication No. WO2014/110218, the content of which is incorporated herein by reference in its entirety.

This application is related to U.S. patent application Ser. No. 14/759,020, filed Jul. 2, 2015, U.S. Publication No. 2015/0342690, the content of which is incorporated herein by reference in its entirety.

This application is related to U.S. Provisional Application No. 61/656,600, filed Jun. 7, 2012, the content of which is incorporated herein by reference in its entirety.

This application is related to PCT Application No. PCT/US2013/043858, filed Jun. 3, 2013, PCT Publication No. WO2013/184560, the content of which is incorporated herein by reference in its entirety.

This application is related to U.S. patent application Ser. No. 14/402,224, filed Nov. 19, 2014, U.S. Publication No. 2015/0150633, the content of which is incorporated herein by reference in its entirety.

This application is related to U.S. Provisional Application No. 61/825,297, filed May 20, 2013, the content of which is incorporated herein by reference in its entirety.

This application is related to PCT Application No. PCT/US2013/038701, filed May 20, 2014, PCT Publication No. WO2014/189876, the content of which is incorporated herein by reference in its entirety.

This application is related to U.S. patent application Ser. No. 14/888,541, filed Nov. 2, 2015, U.S. Publication No. 2016/0074028, now U.S. Pat. No. 9,517,059, issued on Dec. 13, 2016, the content of which is incorporated herein by reference in its entirety.

This application is related to U.S. patent application Ser. No. 15/350,549, filed Nov. 14, 2016, U.S. Publication No. 2017/0119364, now U.S. Pat. No. 10,016,187, issued on Jul. 10, 2018, the content of which is incorporated herein by reference in its entirety.

This application is related to U.S. patent application Ser. No. 16/020,115, filed Jun. 27, 2018, U.S. Publication No. 2018/0368823, the content of which is incorporated herein by reference in its entirety.

This application is related to U.S. Provisional Application No. 61/818,878, filed May 2, 2013, the content of which is incorporated herein by reference in its entirety.

This application is related to PCT Application No. PCT/US2014/036571, filed May 2, 2014, PCT Publication No. WO2014/179683, the content of which is incorporated herein by reference in its entirety.

This application is related to U.S. patent application Ser. No. 14/888,189, filed Oct. 30, 2015, U.S. Publication No. 2016/0067000, now U.S. Pat. No. 9,913,695, issued on Mar. 13, 2018, the content of which is incorporated herein by reference in its entirety.

This application is related to U.S. patent application Ser. No. 15/916,664, filed Mar. 9, 2018, U.S. Publication No. 2018/0256269, the content of which is incorporated herein by reference in its entirety.

This application is related to U.S. Provisional Application No. 61/909,605, filed Nov. 27, 2013, the content of which is incorporated herein by reference in its entirety.

This application is related to U.S. Provisional Application No. 62/052,736, filed Sep. 19, 2014, the content of which is incorporated herein by reference in its entirety.

This application is related to PCT Application No. PCT/US2014/067091, filed Nov. 24, 2014, PCT Publication No. WO2015/081008, the content of which is incorporated herein by reference in its entirety.

This application is related to U.S. patent application Ser. No. 15/038,531, filed May 23, 2016, U.S. Publication No. 2016/0287224, the content of which is incorporated herein by reference in its entirety.

This application is related to U.S. Provisional Application No. 62/008,453 filed Jun. 5, 2014, the content of which is incorporated herein by reference in its entirety.

This application is related to PCT Application No. PCT/US2015/034424, filed Jun. 5, 2015, PCT Publication No. WO2015/188071, the content of which is incorporated herein by reference in its entirety.

This application is related to U.S. patent application Ser. No. 15/315,868, filed Dec. 2, 2016, U.S. Publication No. 2017/0100197, the content of which is incorporated herein by reference in its entirety.

This application is related to U.S. patent application Ser. No. 16/225,156, filed Dec. 19, 2018, U.S. Publication No. 2019/xxxxxx, the content of which is incorporated herein by reference in its entirety.

This application is related to U.S. Provisional Application No. 62/150,223, filed Apr. 20, 2015, the content of which is incorporated herein by reference in its entirety.

This application is related to U.S. Provisional Application No. 62/299,249, filed Feb. 24, 2016, the content of which is incorporated herein by reference in its entirety.

This application is related to PCT Application No. PCT/US2016/028374, filed Apr. 20, 2016, PCT Publication No. WO2016/172162, the content of which is incorporated herein by reference in its entirety.

This application is related to U.S. patent application Ser. No. 15/567,109, filed Oct. 17, 2017, U.S. Publication No. 2018-0228557 the content of which is incorporated herein by reference in its entirety.

This application is related to U.S. Provisional Application No. 62/401,390, filed Sep. 29, 2016, the content of which is incorporated herein by reference in its entirety.

This application is related to PCT Application No. PCT/US2017/054297, filed Sep. 29, 2017, PCT Publication No. WO2018/064475, the content of which is incorporated herein by reference in its entirety.

This application is related to U.S. Provisional Application No. 62/517,433, filed Jun. 9, 2017, the content of which is incorporated herein by reference in its entirety.

This application is related to PCT Application No. PCT/US2018/036876, filed Jun. 11, 2018, PCT Publication No. WO2018/227180, the content of which is incorporated herein by reference in its entirety.

This application is related to U.S. Provisional Application No. 62/481,309, filed Apr. 4, 2017, the content of which is incorporated herein by reference in its entirety.

This application is related to U.S. Provisional Application No. 62/598,812, filed Dec. 14, 2017, the content of which is incorporated herein by reference in its entirety.

This application is related to U.S. Provisional Application No. 62/617,513, filed Jan. 15, 2018, the content of which is incorporated herein by reference in its entirety.

This application is related to PCT Application No. PCT/US2018/026016, filed Apr. 4, 2018, PCT Publication No. WO2018/187425 the content of which is incorporated herein by reference in its entirety.

This application is related to U.S. Provisional Application No. 62/533,644, filed Jul. 17, 2017, the content of which is incorporated herein by reference in its entirety.

This application is related to U.S. Provisional Application No. 62/614,263, filed Jan. 5, 2018, the content of which is incorporated herein by reference in its entirety.

This application is related to PCT Application No. PCT/US2018/042449, filed Jul. 17, 2018, PCT Publication No. WO2019/xxxxxx, the content of which is incorporated herein by reference in its entirety.

This application is related to U.S. Provisional Application No. 62/582,283, filed Nov. 6, 2017, the content of which is incorporated herein by reference in its entirety.

This application is related to U.S. Provisional Application No. 62/614,346, filed Jan. 5, 2018, the content of which is incorporated herein by reference in its entirety.

This application is related to PCT Application No. PCT/US2018/059338, filed Nov. 6, 2018, PCT Publication No. WO2019/xxxxxx, the content of which is incorporated herein by reference in its entirety.

This application is related to U.S. Design Application No. 29/632,148, filed Jan. 5, 2018, the content of which is incorporated herein by reference in its entirety.

This application is related to U.S. Pat. No. 9,011,318, issued Apr. 21, 2015, the content of which is incorporated herein by reference in its entirety.

### BACKGROUND

As less invasive medical techniques and procedures become more widespread, medical professionals such as surgeons may require articulating surgical tools, such as endoscopes, to perform such less invasive medical techniques and procedures that require access to locations within the patient, such as a site accessible through the mouth or other natural orifice, or a site accessible through an incision through the patient's skin.

There is a need for improved systems for performing a medical procedure.

### SUMMARY

In an aspect, a system for performing a medical procedure on a patient comprises: an articulating probe assembly, comprising: an inner probe comprising multiple articulating inner links; an outer probe surrounding the inner probe and comprising multiple articulating outer links; and at least two working channels that exit a distal portion of the probe assembly; at least one tool configured to translate through one of the at least two working channels; and a manipulator for controlling the at least one tool.

In an embodiment, the manipulator is constructed and arranged to continuously rotate the at least one tool.

In an embodiment, the at least one tool comprises an inner part and an outer part.

In an embodiment, the manipulator is constructed and arranged to continuously rotate the inner part of the at least one tool.

In an embodiment, the manipulator is constructed and arranged to continuously rotate the outer part of the at least one tool.

In an embodiment, the manipulator is constructed and arranged to continuously rotate the inner part and the outer part of the at least one tool.

In an embodiment, the manipulator comprises at least one motor.

In an embodiment, the at least one motor is in an inner rotation frame.

In an embodiment, the at least one motor comprises a plurality of motors that are constructed and arranged in a radial pattern.

In an embodiment, the manipulator comprises at least one controller that is constructed and arranged to control the at least one motor.

In an embodiment, the at least one controller is directly attached to the at least one motor.

In an embodiment, the manipulator comprises a scaffolding structure.

### BRIEF DESCRIPTION OF THE DRAWINGS

The foregoing and other objects, features and advantages of embodiments of the present inventive concepts will be apparent from the more particular description of preferred embodiments, as illustrated in the accompanying drawings in which like reference characters refer to the same elements throughout the different views. The drawings are not necessarily to scale, emphasis instead being placed upon illustrating the principles of the preferred embodiments.

FIG. 1 is a schematic view of a system in which embodiments of the present inventive concepts can be practiced.

FIGS. 1A-C are graphic demonstrations of a robotic probe, in accordance with embodiments of the present inventive concepts.

FIG. 2A is a perspective view of a tool drive, in accordance with embodiments of the present inventive concepts.

FIG. 2B is a perspective cross-sectional view of an outer support assembly, outer rotating assembly, and inner rotating assembly of a tool drive, in accordance with embodiments of the present inventive concepts.

FIG. 3A is a perspective view of an inner rotating assembly, in accordance with embodiments of the present inventive concepts.

FIG. 3B is a perspective cross-sectional view of an inner rotating assembly, in accordance with embodiments of the present inventive concepts.

FIG. 4A is a perspective view of an outer rotating assembly, in accordance with embodiments of the present inventive concepts.

FIG. 4B is a perspective cross-sectional view of an outer rotating assembly, in accordance with embodiments of the present inventive concepts.

FIG. 5A is a perspective view of an outer support assembly, in accordance with embodiments of the present inventive concepts.

FIG. 5B is a perspective cross-sectional view of an outer support assembly, in accordance with embodiments of the present inventive concepts.

FIG. 6A is a perspective view of a tool drive including a concentrically positioned outer support assembly, outer rotating assembly, and inner rotating assembly along with an interface assembly, in accordance with embodiments of the present inventive concepts.

FIG. 6B is a perspective view of an interface assembly for connecting a tool to an interface assembly, in accordance with embodiments of the present inventive concepts.

FIG. 7 is a perspective view of a linear drive assembly, in accordance with embodiments of the present inventive concepts.

### DETAILED DESCRIPTION OF EMBODIMENTS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate” shall include locations relatively close to, on, in and/or within a referenced component, anatomical location, or other location.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence.

The term “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

In this specification, unless explicitly stated otherwise, “and” can mean “or,” and “or” can mean “and.” For example, if a feature is described as having A, B, or C, the feature can have A, B, and C, or any combination of A, B, and C. Similarly, if a feature is described as having A, B, and C, the feature can have only one or two of A, B, or C.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

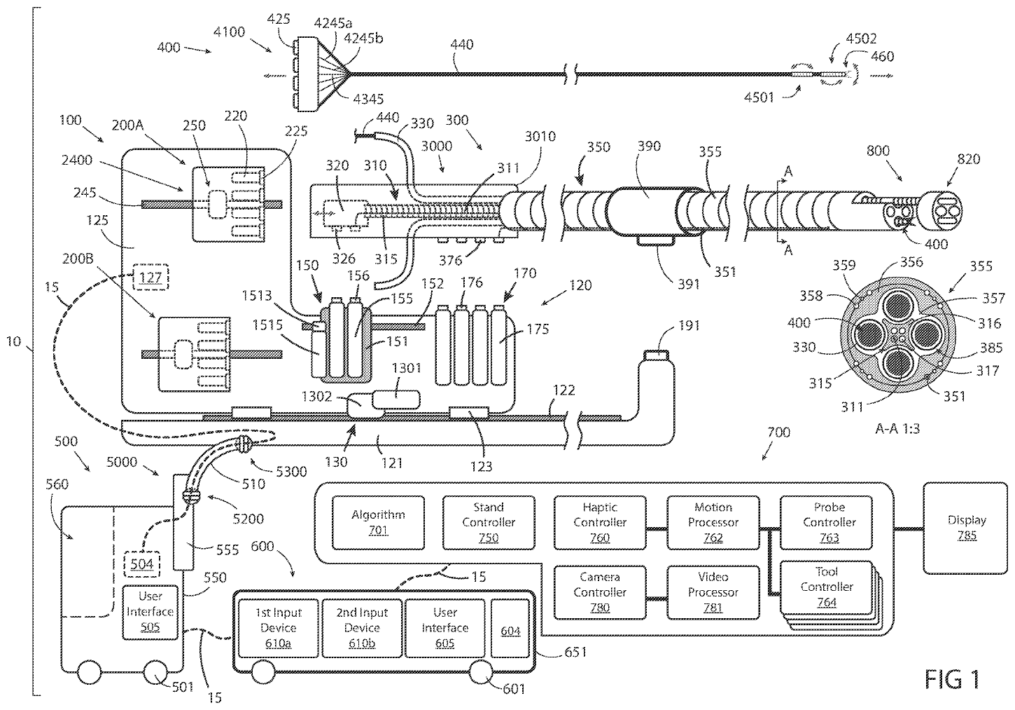

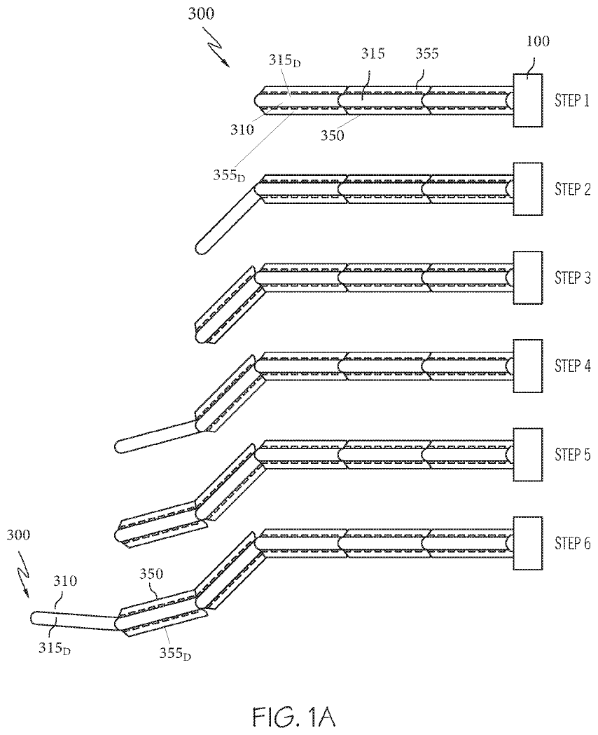

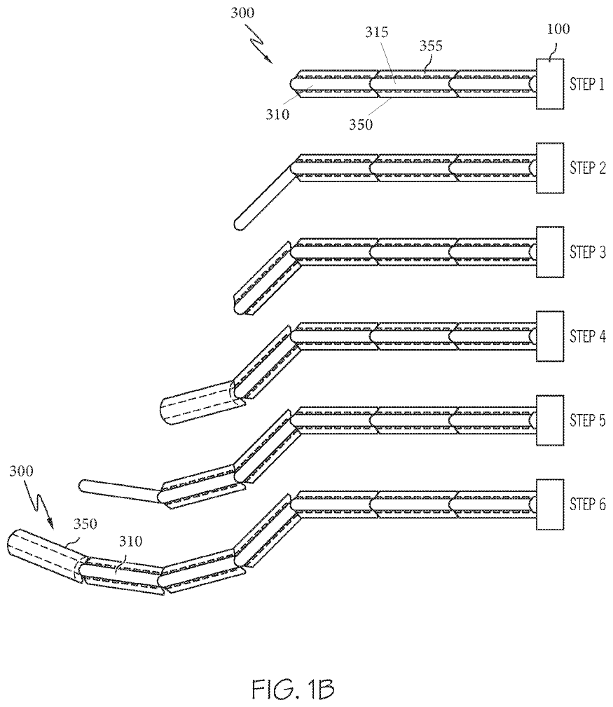

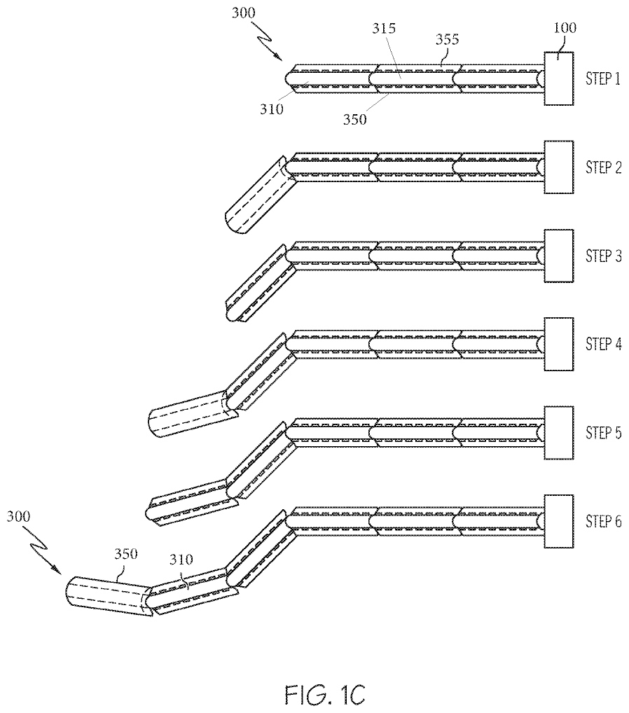

Referring to FIG. 1, a schematic view of a system in which embodiments of the present inventive concepts can be practiced is illustrated.

System 10 includes a robotic feeder 100. Feeder 100 interchangeably and operably engages a robotic probe assembly 300, and at least one robotic tool assembly 400. Feeder 100 is constructed and arranged to advance, retract, steer, and/or otherwise control the position and/or articulation of probe assembly 300 and/or tools 400, as described herein. One or more tools 400 can be slidingly received within a channel of probe assembly 300, and each tool 400 can be advanced beyond the distal end of probe assembly 300. Feeder 100 includes a probe manipulation assembly 120 for operably controlling the position and articulation of probe assembly 300. Feeder 100 also includes at least one tool manipulation assembly, tool drive 200 (e.g. tool drives 200A and 200B shown), for controlling the position and articulation of an attached tool 400. System 10 further includes a multi-dimensional positioning assembly, stand 500. Stand 500 includes an articulation assembly 5000 for positioning feeder 100 with multiple degrees of freedom, for example within an operating room, relative to a patient and/or patient bed, as described herein. System 10 further includes a control interface, surgeon console 600, configured to receive commands from one or more operators of system 10 (e.g. one or more surgeons or other clinicians). Console 600 can include a first and second input device, 610A and 610B respectively (singly or collectively input devices 610 herein), each configured to receive multi-dimensional input data (e.g. via a kinematic input device as described herein). System 10 further includes a collection of data processing components, collectively processing unit 700. Processing unit 700 can include one or more algorithms, controllers, memory, state machines, and/or processors, singly and/or collectively controlling one or more components of system 10 (e.g. based at least on one or more user inputs received by one or more input components of system 10). System 10 further includes an imaging device, camera assembly 800 (e.g. a tool 400 configured as a camera, as described herein), comprising one or more cameras, camera 820. Image data (e.g. still and/or video images) captured by camera 820 can be displayed on one or more monitors or other screens, display 785. One or more components described herein as included in a tool 400 can also be included in camera assembly 800, for example camera assembly 800 can comprise a tool 400 with camera 820 operably attached thereto. A conduit, bus 15, can connect one or more components of system 10. Bus 15 can comprise one or more electrical, fluid, optical, and/or other conduits for transferring information, power, one or more fluids, light energy, and combinations of one or more of these.

### Probe Assembly 300

Probe assembly 300 includes an outer probe 350, comprising multiple articulating outer links 355. Links 355 each comprise a ring-like structure (e.g. a hollow tube-like structure), link body 356, surrounding a hollow bore, channel 357. Collectively, channels 357 define a lumen extending along at least a portion of the length of outer probe 350. Links 355 can include multiple lumens extending therethrough, such as lumens extending along the link, through link body 356. For example, links 355 can include one or more steering cable lumens, lumens 358, such as eight lumens 358 shown. Lumens 358 can each slidingly receive a steering cable 351 that is used to control at least the articulation of outer probe 350, as described herein. Links 355 can also include one or more auxiliary lumens, four lumens 359 shown. In some embodiments, lumens 359 can slidingly receive elongate devices and/or filaments, such as optical fibers for delivering light to a surgical site.

Probe assembly 300 further includes inner probe 310, comprising multiple articulating inner links 315. Inner probe 310 is slidingly received within channels 356 extending through outer probe 350. Links 315 can comprise a link body 316, and can include multiple lumens extending therethrough, such as lumens extending along the link. For example, links 315 can include one or more steering cable lumens, lumens 317, such as four lumens 318 shown. Lumens 317 can each slidingly receive a steering cable 311 used to control at least the articulation of inner probe 310, as described herein.

The outer shape of link body 316 can align with the shape of the channel 357 to form a plurality of passageways or working channels 385, extending throughout probe assembly 300. Working conduits 330 can be slidingly received within channels 385, extending throughout the probe assembly 300. Each conduit 330 can sliding receive at least a portion of a tool 400.

Probe assembly 300 can be of similar construction and arrangement to the similar device described in reference to applicant's co-pending U.S. patent application Ser. No. 16/114,681, filed Aug. 28, 2018, the content of which is incorporated herein by reference in its entirety.

Probe assembly 300 further comprises a manipulation assembly 3000, operably attached to the proximal portion of probes 310, 350. Manipulation assembly 3000 comprises a housing 3010, surrounding at least a cart 320, operably attached to inner probe 310. Manipulation assembly 3000 comprises one or more bobbins 376 operably attached to one or more steering cables 351 (also referred to herein as control cables). Cart 320 comprises one or more bobbins 326 operably attached to one or more steering cables 311. Manipulation assembly 3000 is constructed and arranged to operably and removably attach to feeder 100, as described herein. Manipulation assembly 3000 supports the proximal sections of one or more working conduits 330 in an orientation that is radially dispersed relative to the radially compact orientation of the distal portions of working conduits 330 within probe assembly 300.

Probe assembly 300 can include a support structure, introducer 390. Introducer 390 can comprise a rigid elongate structure. Introducer 390 can surround at least a portion of probe assembly 300. Introducer 390 can comprise a connector portion 391, constructed and arranged to operably attach to a portion of feeder 100 as described herebelow. Probe assembly 300 can be of similar construction and arrangement to the similar device described in applicant's co-pending application U.S. Provisional Application No. 62/614,240, filed Jan. 5, 2018, the content of which is incorporated herein by reference in its entirety.

### Feeder 100

Feeder 100 comprises a manipulation assembly 120 comprising a carriage 125 operably attached to a base 121. Carriage 125 can comprise one or more linear bearings 123 fixedly attached thereto, slidingly attached to a linear rail assembly 122, which in turn is fixedly attached to base 121. Linear rail assembly 122 can comprise one or more rails and/or lead screws. Manipulation assembly 120 can comprise a linear drive assembly 130, that is operably attached to carriage 125 and linear rail assembly 122. For example, linear rail assembly 122 can comprise at least a lead screw, and linear drive assembly 130 can comprise a motor 1301 and gear box 1302. Linear drive assembly 130 can be configured to engage the lead screw of linear rail assembly 122, such as to translate carriage 125 relative to base 121.

Manipulation assembly 120 can comprise a probe support assembly 170. Probe support assembly 170 can comprise at least a portion of carriage 125. Probe support assembly 170 can comprise one or more motors 175, each operably attached to a capstan 176. Probe support assembly 170 is constructed and arranged to operably and removably attach to manipulation assembly 3000, for example, such that each capstan 176 operably engages a corresponding bobbin 376. Motors 175 can be configured to rotate capstans 176, which in turn rotate bobbins 376, tensioning and de-tensioning cables 351 to control the articulation of outer probe 350.

Probe support assembly 170 can further comprise a probe translation assembly 150. Probe translation assembly 150 can comprise one or more motors 155, each operably attached to a capstan 156. Probe translation assembly 150 is constructed and arranged to operably and removably attach to cart 320, for example such that each capstan 156 operably engages a corresponding bobbin 326. Motors 155 can be configured to rotate capstans 156, which in turn rotate bobbins 326, tensioning and de-tensioning cables 311 to control the articulation of inner probe 310. Probe translation assembly 150 can comprise a cart 151. Motors 155 can be fixedly attached to cart 151. Cart 151 can be slidingly attached to a linear rail assembly 152, fixedly attached to carriage 125. Linear rail assembly 152 can comprise one or more rails and/or lead screws. Probe translation assembly 150 can comprise a motor 1515 and drive gear 1513 operably attached thereto. Drive gear 1513 can operably attach to linear rail assembly 152, for example when linear rail assembly 152 comprises at least a lead screw. Motor 1515 can be configured to rotate drive gear 1513 to translate cart 151 relative to carriage 125. Cart 151 can be constructed and arranged to engage cart 320, such that translation of cart 151 causes the translation of cart 320 within manipulation assembly 3000. Translation of cart 320 can cause the translation of inner probe 310 with respect to outer probe 350, as described herein.

Feeder 100 can include a connector portion 191, constructed and arranged to removably connect to introducer 390 of probe assembly 300. Connector portion 191 can be positioned at the distal end of carriage 125, as shown.

Feeder 100 can include one or more modules 127, such as one or more processors and/or controllers. Module 127 can be operably attached to one or more components of system 10 via bus 15.

Feeder 100 can be of similar construction and arrangement to the similar device described in applicant's co-pending application U.S. Provisional Application No. 62/614,240, filed Jan. 5, 2018, the content of which is incorporated herein by reference in its entirety.

### Tool Drive 200

Each tool drive 200 (also referred to herein as a singular tool drive 200) is configured to operably and interchangeably attach to one or more tools 400. Feeder 100 can comprise one, two, three, four, or more tool drives, tool drives 200A and 200B shown. Additional tool drives can be mounted to carriage 125 opposite tool drives 200A and 200B (e.g. on the opposite side of carriage 125). Tool drive 200 can slidingly attach to carriage 125 via a translation assembly 2400. Translation assembly 2400 can comprise a linear rail assembly 245, fixedly attached to carriage 125. Linear rail assembly 245 can comprise one or more rails and/or lead screws. Translation assembly 2400 can further comprise a linear drive assembly 250, operably attached to tool drive 200 and linear rail assembly 245. For example, linear rail assembly 245 can comprise at least a lead screw, and linear drive assembly 250 can comprise a motor and/or a gear box. Linear drive assembly 250 can be configured to engage the lead screw of linear rail assembly 245, to translate tool drive 200 relative to carriage 125. Translation of tool drive 200 can cause the translation of an attached tool 400, for example relative to outer probe 350 operably attached to manipulation assembly 120.

Tool drive 200 can comprise one or more motors 220, configured to manipulate one or more components of tool drive 200. For example, one or more motors 220 can be configured to rotate one or more assemblies of tool drive 200 relative to each other, and/or to rotate one or more gears 225 (e.g. capstans) of tool drive 200. Gears 225 of tool drive 200 can be configured to operably engage one or more bobbins of an attached tool 400, as described herein, to control the articulation of the attached tool 400.

Tool drive 200 can be of similar construction and arrangement to the similar device described in applicant's co-pending application U.S. Provisional Application No. 62/614,228, filed Jan. 5, 2018, the content of which is incorporated herein by reference in its entirety.

### Tool 400

Tool 400 can include a manipulation assembly 4100, operably attached to the proximal end of a shaft 440. Shaft 440 can comprise a flexible shaft, comprising one or more lumens. Tool 400 can comprise one or more sets of steering (or control) cables 4245a, 4245b, and or 4345. Cables 4245a,b can be operably attached to manipulation assembly 4100, and extend through shaft 440 to a first and second articulation section 4501 and 4502, respectively. Cables 4245a,b can be tensioned and/or de-tensioned by manipulation assembly 4100 to cause the articulation of articulation sections 4501 and 4502, respectively. Cables 4345 can be operably attached to manipulation assembly 4100, and extend through shaft 440 to an end effector 460. Cables 4345 can be tensioned and/or de-tensioned by manipulation assembly 4100 to cause the articulation or other manipulation of end effector 460. System 10 can comprise multiple tools 400, such as four, five, six, or more tools 400, each exchangeable and operably attachable to tool drives 200. End effectors 460 can comprise scissors, graspers, blades, cautery devices, laser devices, and the like. Manipulation assembly 4100 can be constructed and arranged to removably attach to tool drive 200, such that gears 225 engage bobbins 425 of manipulation assembly 4100. Motors 220 of tool drive 200 can rotate gears 225, and bobbins 425, to tension and/or de-tension one or more cables of tool 400 described herein, to tension and/or de-tension the cables and manipulate tool 400. Manipulation assembly 4100 can also be constructed and arranged to rotate one or more components of tool 400 relative to each other, for example to rotate end effector 460 relative to shaft 440.

Tool 400 can be of similar construction and arrangement to the similar device described in applicant's co-pending application U.S. Provisional Application No. 62/614,225, filed Jan. 5, 2018, the content of which is incorporated herein by reference in its entirety.

### Camera Assembly 800

In some embodiments, as described hereabove, a tool 400 can be configured as a camera assembly 800. Camera assembly 800 can comprise a camera 820, operably attached to the distal end of shaft 440 of a tool 400. In some embodiments, camera 820 is attached to shaft 440 after shaft 440 has been inserted through probe assembly 300. For example, in some embodiments, camera 820 is larger than working channel 385.

Camera assembly 800 can be of similar construction and arrangement to the similar device described in applicant's co-pending application PCT International Patent Application No. PCT/US2018/059338, filed Nov. 6, 2018, the content of which is incorporated herein by reference in its entirety.

### Stand 500

Stand 500 can be constructed and arranged to position feeder 100 relative to a patient and/or patient bed, such as to position probe assembly 300 for a surgical procedure. For example, surgical procedures can include but are not limited to transabdominal procedures, transoral procedures, trans anal procedures, and/or trans vaginal procedures. Stand 500 includes a base 550, supporting an articulation assembly 5000. Articulation assembly 5000 includes a tower 555, extending vertically from base 550. A first hub 5200 is operably attached to tower 555. First hub 5200 can be adjusted along the height of tower 555, via one or more motors and/or vertical translation assemblies. First hub 5200 is operably attached to positioning arm 510, which is operably attached to a second hub 5300. Second hub 5300 is operably attached to base 121 of feeder 100. Hubs 5200 and 5300 can each comprise one or more motors, gears, hinges, axles, and the like, configured to manipulate the position of feeder 100 relative to stand 500. Bus 15 of system 10 can operably connect feeder 100 to stand 500. In some embodiments, bus 15 is routed through hubs 5200, 5300, arm 510, and/or tower 555, such that bus 15 is at least partially contained within articulation assembly 5000.

Stand 500 can comprise a recess 560. Articulation assembly 5000 can be configured to “fold” into a stowed position, with feeder 100 positioned at least partially within recess 560. In some embodiments stand 500 can comprise a processor 504 and a user interface 505. User interface 505 can include input and output functionality, such as a touchscreen monitor. User interface 505 can be configured to allow a user to control one or more components of system 10, for example the articulation of articulation assembly 5000. In some embodiments, stand 500 includes one or more wheels 501, and is constructed and arranged to be mobile. For example, stand 500 can be manually repositionable by a user and/or can be robotically repositionable, for example when wheels 501 are driven by one or more motors.

Stand 500 can be of similar construction and arrangement to the similar device described in applicant's co-pending application U.S. Provisional Application No. 62/614,223, filed Jan. 5, 2018, the content of which is incorporated herein by reference in its entirety.

### Surgeon Console 600

Surgeon console 600 can be operably attached to one or more components of system 10, such as via bus 15. Console 600 can comprise a base 651, supporting input devices 610a,b, and user interface 605. Console 600 can comprise a processor 604. In some embodiments, processor 604 can receive commands from input device 610a,b, and/or user interface 605. User interface 605 can be configured to allow a user to control one or more components of system 10. In some embodiments, user interface 605 can be a redundant interface of user interface 505, such that a user can perform the same operations from either interface. In some embodiments, console 600 includes one or more wheels 601, and is constructed and arranged to be mobile. For example, console 600 can be manually repositionable by a user and/or can be robotically repositionable, for example when wheels 601 are driven by one or more motors.

Console 600 can be of similar construction and arrangement to the similar device described in applicant's co-pending application U.S. Provisional Application No. 62/614,224, filed Jan. 5, 2018, the content of which is incorporated herein by reference in its entirety.

### Processor 700

Processing unit 700 can comprise one or more controllers and/or processors, located throughout system 10. For example, processor 700 can comprise a computer or other processing device, and/or can comprise one or more controllers or modules of system 10 (e.g. module 127 of feeder 100, processor 504 of stand 500, and/or processor 604 of user interface 600). Processing unit 700 can comprise one or more algorithms for processing data and/or commanding one or more components of system 10 to perform one or more operations. Processing unit 700 can comprise one or more controllers for controlling components of system 10. Processing unit 700 can comprise a stand controller 750, for operational control of stand 500. Processing unit 700 can comprise a camera controller, for operational control of camera assembly 800. Camera controller 780 can be operably attached to a video processor 781 for processing image data captured by camera 820. Video processor 781 can provide processed image data to a display 785, for display to a user. Processing unit 700 can comprise a haptic controller 760, operably attached to input devices 610a,b of console 600, for example via processor 604. Haptic controller 760 can be operably attached to a motion processor 762, which is operably attached to a probe controller 763, and one or more tool controllers 764. Haptic controller 760 can receive multi-dimensional input data (e.g. via a kinematic input device) from input devices 610a,b, and/or provide haptic feedback commands to input devices 610a,b. Motion processor 762 can process the multi-dimensional input data, and provide articulation and/or translation commands to probe controller 763 and/or tool controllers 764. Probe controller 763 can provide commands to one or more motors of system 10, for example to one or more motors of manipulation assembly 120 to at least advance, retract, steer, and/or otherwise control the position and/or articulation of probe assembly 300. Tool controllers 764 can provide commands to one or more motors of system 10, for example one or more motors of a tool drive 200 to at least advance, retract, steer, and/or otherwise control the position and/or articulation of an attached tool 400.

Processor 700 can be of similar construction and arrangement to the similar device described in applicant's co-pending application U.S. Provisional Application No. 62/614,235, filed Jan. 5, 2018, the content of which is incorporated herein by reference in its entirety.

Referring additionally to FIGS. 1A-C, graphic demonstrations of a robotic probe 300 are illustrated, consistent with the present inventive concepts. Articulating probe 300 comprises essentially two concentric mechanisms, an outer mechanism and an inner mechanism, each of which can be viewed as a steerable mechanism. Each of the components of probe 300 can comprise one or more sealing elements, such as to support an insufflation procedure. FIGS. 1A-C show the concept of how different embodiments of robotic probe 300 operate. Referring to FIG. 1A, the inner mechanism can be referred to as a first mechanism or inner probe 310. The outer mechanism can be referred to as a second mechanism or outer probe 350. Each mechanism can alternate between rigid and limp states. In the rigid mode or state, the mechanism is just that—rigid. In the limp mode or state, the mechanism is highly flexible and thus either assumes the shape of its surroundings or can be re-shaped. It should be noted that the term “limp” as used herein does not necessarily denote a structure that passively assumes a particular configuration dependent upon gravity and the shape of its environment; rather, the “limp” structures described in this application are capable of assuming positions and configurations that are desired by the operator of the device, and therefore are articulated and controlled rather than flaccid and passive.

In some embodiments, one mechanism starts limp and the other starts rigid. For the sake of explanation, assume outer probe 350 is rigid and inner probe 310 is limp, as seen in step 1 in FIG. 1A. Now, inner probe 310 is both pushed forward by feeder 100, and a distal-most inner link 315D is steered, as seen in step 2 in FIG. 1A. Now, inner probe 310 is made rigid and outer probe 350 is made limp. Outer probe 350 is then pushed forward until a distal-most outer link 355D catches up to the distal-most inner link 315D (e.g. outer probe 350 is coextensive with inner probe 310), as seen in step 3 in FIG. 1A. Now, outer probe 350 is made rigid, inner probe 310 limp, and the procedure then repeats. One variation of this approach is to have outer probe 350 be steerable as well. The operation of such a device is illustrated in FIG. 1B. In FIG. 1B it is seen that each mechanism is capable of catching up to the other and then advancing one link beyond. According to one embodiment, outer probe 350 is steerable and inner probe 310 is not. The operation of such a device is shown in FIG. 1C.

In medical applications, operation, procedures, and so on, once robotic probe 300 arrives at a desired location, the operator, such as a surgeon, can slide one or more tools through one or more working channels of outer probe 350, inner probe 310, or one or more working channels formed between outer probe 350 and inner probe 310, such as to perform various diagnostic and/or therapeutic procedures. In some embodiments, the channel is referred to as a working channel that can, for example, extend between first recesses formed in a system of outer links and second recesses formed in a system of inner links. Working channels may be included on the periphery of robotic probe 300, such as working channels comprising one or more radial projections extending from outer probe 350, these projections including one or more holes sized to slidingly receive one or more tools. As described with reference to other embodiments, working channels may be positioned on other locations extending from, on, in, and/or within robotic probe 300.

Inner probe 310 and/or outer probe 350 are steerable and inner probe 310 and outer probe 350 can each be made both rigid and limp, allowing robotic probe 300 to drive anywhere in three-dimensions while being self-supporting. Articulating probe 300 can “remember” each of its previous configurations and for this reason, robotic probe 300 can retract from and/or retrace to anywhere in a three-dimensional volume such as the intracavity spaces in the body of a patient such as a human patient.

Inner probe 310 and outer probe 350 each include a series of links, i.e. inner links 315 and outer links 355 respectively, that articulate relative to each other. In some embodiments, outer links 355 are used to steer and lock robotic probe 300, while inner links 315 are used to lock robotic probe 300. In a “follow the leader” fashion, while inner links 315 are locked, outer links 355 are advanced beyond the distal-most inner link 315D. Outer links 355 are steered into position by the system steering cables, and then locked by locking the steering cables. The cable of inner links 315 is then released and inner links 315 are advanced to follow outer links 355. The procedure progresses in this manner until a desired position and orientation are achieved. The combined inner links 315 and outer links 355 may include working channels for temporary or permanent insertion of tools at the surgery site. In some embodiments, the tools can advance with the links during positioning of robotic probe 300. In some embodiments, the tools can be inserted through the links following positioning of robotic probe 300.

One or more outer links 355 can be advanced beyond the distal-most inner link 315D prior to the initiation of an operator controlled steering maneuver, such that the quantity extending beyond the distal-most inner link 315D will collectively articulate based on steering commands. Multiple link steering can be used to reduce procedure time, such as when the specificity of single link steering is not required. In some embodiments, between 2 and 20 outer links can be selected for simultaneous steering, such as between 2 and 10 outer links or between 2 and 7 outer links. The number of links used to steer corresponds to achievable steering paths, with smaller numbers enabling more specificity of curvature of robotic probe 300. In some embodiments, an operator can select the number of links used for steering (e.g. to select between 1 and 10 links to be advanced prior to each steering maneuver).

In some embodiments, the system 10 provides unlimited continuous rotation of a tool 400, such as to accommodate and promote surgical applications such as suturing and knot tying.

In some embodiments, the system 10 provides unlimited continuous rotation of the inner and the outer part of a tool 400, such as to enable precise rotation of a tool 400 tip. In some embodiments, the motors (e.g. actuators) used for actuating the tool 400 tip are positioned within the inner rotation frame, such as to maintain constant tension on closed-line cables that drive the tool 400 tip.

In some embodiments, the motors are arranged in a radial pattern that provides a compact form factor. For example, with four tool drives 200 in system 10, a compact form factor is advantageous to reduce the overall system size.

In some embodiments, the design employs a scaffolding structure, that supports the rotating members (the inner rotation and outer rotation) at both ends using bearings. The scaffolding structure provides the necessary rigidity while reducing the weight of the system. For example, with four tool drives 200 in system 10, the resulting reduced weight is advantageous.

In some embodiments, the controllers used for controlling the motors are attached directly to the motors to simplify the cable management. For example, for 15 motors with 12 cables coming out of each motor, cable management is not a trivial challenge. A CAN bus can be used to communicate with the controllers. The CAN bus and power bus passes through a slip ring to provide continuous rotation. Following this scheme, the total number of cables coming out of each motor is reduced to just 5 cables (3 cables for CAN and 2 cables for power).

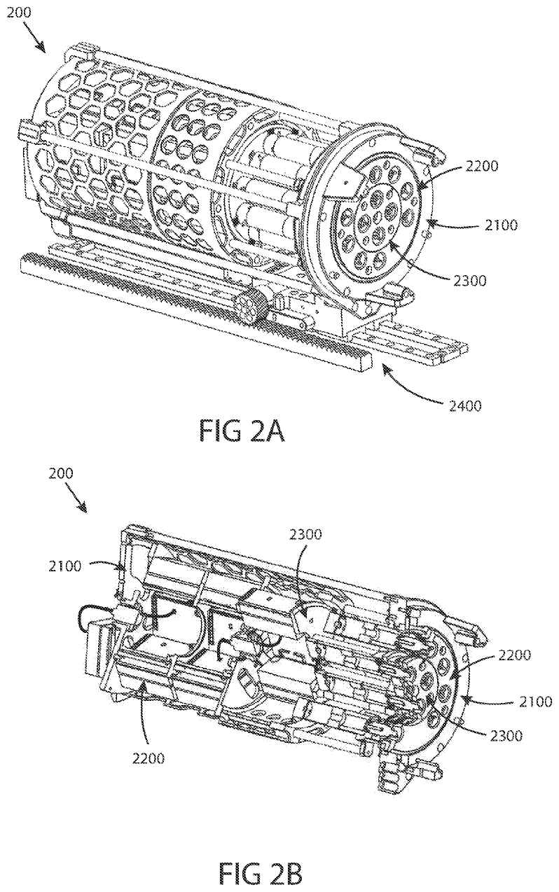

Referring to FIG. 2A, a perspective view of a tool drive 200 is illustrated, in accordance with embodiments of the present inventive concepts. In some embodiments, a tool drive 200 comprises an outer support assembly 2100, an outer rotating assembly 2200, and an inner rotating assembly 2300. Outer support assembly 2100 can be operably coupled to a linear drive assembly 2400. In some embodiments, the outer rotating assembly 2200 is rotatably positioned within the outer support assembly 2100. In some embodiments, the inner rotating assembly 2300 is rotatably positioned within the outer rotating assembly 2200.

In some embodiments, the outer support assembly 2100 is operably attached to the linear drive assembly 2400. The linear drive assembly 2400 can be fixedly attached to a frame (not shown), and can be configured to translate the outer support assembly 2100 along the linear drive assembly 2400. In some embodiments, the linear drive assembly is attached to carriage 125 of FIG. 1.

Referring additionally to FIG. 2B, a perspective cross-sectional view of the outer support assembly 2100, the outer rotating assembly 2200, and the inner rotating assembly 2300 of the tool drive 200 is illustrated, in accordance with embodiments of the present inventive concepts. In FIG. 2B, the linear drive assembly 2400 has been removed for illustrative clarity.

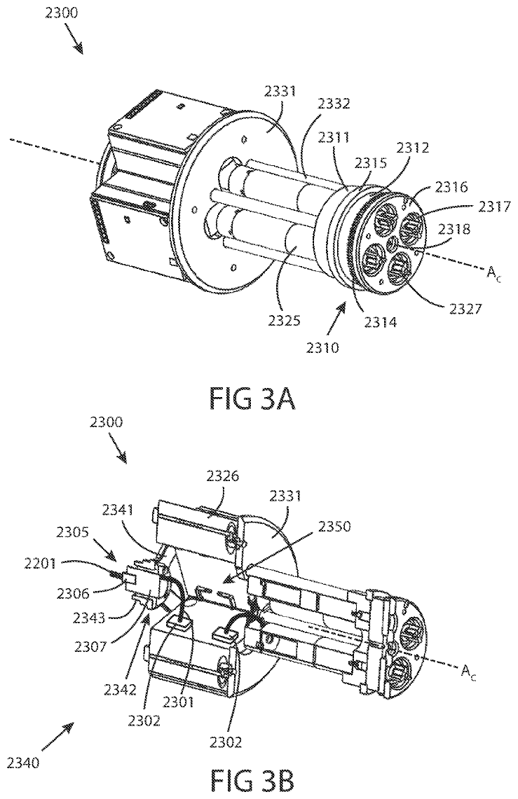

In FIG. 2B, the inner rotating assembly 2300 is illustrated as being rotated in a counter-clockwise direction, and the outer rotating assembly 2200 is illustrated as being rotated in a clockwise direction. For purposes of the description of the present embodiment, the term counter-clockwise refers to a direction of rotation wherein a top portion of the rotating assembly is rotated out of the page and the term clockwise refers to a direction of rotation wherein a top portion of the rotating assembly is rotated into the page. Inner rotating assembly 2300 and outer rotating assembly 2200 are described in further detail herebelow in reference to FIGS. 3 through 7.

Referring to FIG. 3A, a perspective view of the inner rotating assembly 2300 is illustrated, in accordance with embodiments of the present inventive concepts. Referring additionally to FIG. 3B, a perspective cross-sectional view of the inner rotating assembly 2300 is illustrated, in accordance with embodiments of the present inventive concepts. In some embodiments, the inner rotating assembly 2300 comprises a cylindrical structure that has a relatively symmetrical and/or otherwise balanced construction, about central Axis A_{C}. In some embodiments, the inner rotating assembly 2300 rotates about central Axis A_{C}.

In some embodiments, the inner rotating assembly 2300 comprises a distal support assembly 2310. The distal support assembly 2310 can comprise a first plate 2311 and a second plate 2312. In some embodiments, the first plate 2311 and the second plate 2312 of the distal support assembly 2300 mate to capture a cylindrical bearing, bearing 2315. In some embodiments, the bearing 2315 is attached to the distal support assembly 2310 via a press fit relationship. In some embodiments, the bearing 2315 is attached to the distal support assembly 2310 with one or more screws. In some embodiments, the bearing 2315 is attached to the distal support assembly 2310 with glue, or otherwise fixedly attached to the distal support assembly 2310

In some embodiments, the distal support assembly 2310 further includes gear 2314. Gear 2314 can operably engage a mating gear of the outer rotating assembly 2200, described herein, to rotate the inner rotating assembly 2300 relative to the outer rotating assembly 2200 when driven by a motor of the outer rotating assembly 2200.

In some embodiments, the distal support assembly 2310 further includes a distal cover plate 2316. Distal support assembly 2310 can comprise one or more holes 2317, extending through the first plate 2311, the second plate 2312, cover plate 2316, and/or gear 2314. In the embodiment shown in FIG. 3A, four holes 2317 are shown equally spaced about the circumference of the distal support assembly 2310. In some embodiments, the distal support assembly 2310 further comprises a center hole 2318 for example, positioned along central axis A_{C}. In some embodiments, the distal support assembly 2310 is fixedly attached to a medial support structure 2331, by one or more standoffs, 2332.

Inner rotating assembly 2300 comprises one or more motors 2325, such as four motors 2325 shown in FIG. 3A. In some embodiments, rotating assembly 2300 comprises less than four motors 2325. In other embodiments, rotating assembly 2300 comprises more than four motors 2325. The motors 2325 can be fixedly attached to the distal support assembly 2310 and positioned between the distal support assembly 2310 and the medial support structure 2331. Each motor 2325 is operably attached to a rotatable gear 2327, and each rotatable gear 2327 is positioned within a hole 2317.

In some embodiments, the inner rotating assembly 2300 comprises one or more motor controller assemblies 2326, each fixedly attached to the proximal side of the medial support structure 2331 and operably attached to the motors 2325. The one or more motor controller assemblies 2326 can provide power and/or control signals to motors 2325, and/or can monitor motors 2325. In some embodiments, the one or more controller assemblies 2326 are configured to monitor one or more parameters that include, but are not limited to, the current, the heat, and the rotational position of the motors of the motors 2325 via one or more busses 2301. In some embodiments, the motors comprise digital motors, closed-loop motors, or closed-loop servo-motors.

In some embodiments, the inner rotating assembly 2300 further includes a proximal support assembly 2340. The proximal support assembly 2340 can include one or more struts 2341 and a supporting hub 2342. The one or more struts 2341 can be fixedly attached to the proximal sides of the motor control assemblies 2326.

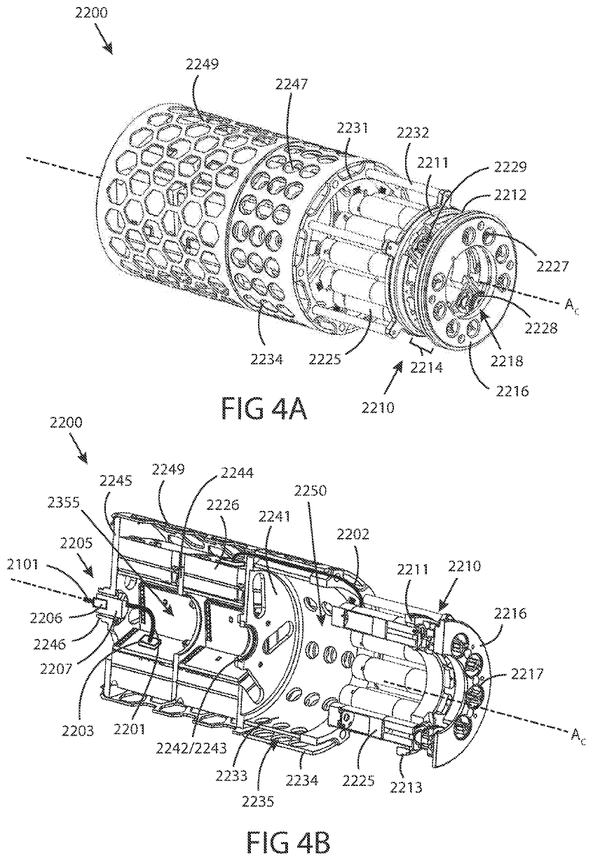

In some embodiments, the supporting hub 2342 includes a bearing surface 2343 that rotatably engages a corresponding bearing surface 2243 of the outer rotating assembly 2200, as described herebelow in reference to FIGS. 4A and B.

In some embodiments, the supporting hub 2342 supports a slip ring 2305. In some embodiments, the slip ring 2305 rotatably and electrically couples a bus 2201 of the outer rotating assembly 2200 to a bus 2301 of the inner rotating assembly 2300. A proximal portion 2306 of the slip ring 2305 can rotate relative to a distal portion 2307, which rotates with the inner rotating assembly 2300, while maintaining one or more electrical connections between bus 2201 and bus 2301. In some embodiments, bus 2301 daisy chains to each motor control assembly 2326, which each operably attach to motors 2325.

The motor control assemblies 2326, along with the medial support structure 2331 and the one or more struts 2341, define an inner volume, chamber 2350, within which the buses 2301 (e.g. wires) are routed and connect to the motor control assemblies 2326, such as via connectors 2302.

Referring to FIG. 4A, a perspective view of the outer rotating assembly 2200 is illustrated, in accordance with embodiments of the present inventive concepts. Referring additionally to FIG. 4B, a perspective cross-sectional view of the outer rotating assembly 2200 is illustrated, in accordance with embodiments of the present inventive concepts. In some embodiments, the outer rotating assembly 2200 comprises a cylindrical structure, in a primarily symmetrical and/or otherwise balanced construction, about central axis A_{C}. In some embodiments, the outer rotating assembly 2200 rotates about central axis A_{C}. In some embodiments, the outer rotating assembly 2200 comprises a distal support assembly 2210. The distal support assembly 2210 can comprise a first ring 2211 and a second ring 2212. The first ring 2211 and the second ring 2212 can be offset using standoffs (or other methods), to define a space or gap 2214 between them. One or more gears 2228, 2229, as described herebelow, can be positioned at least partially within the gap 2214.

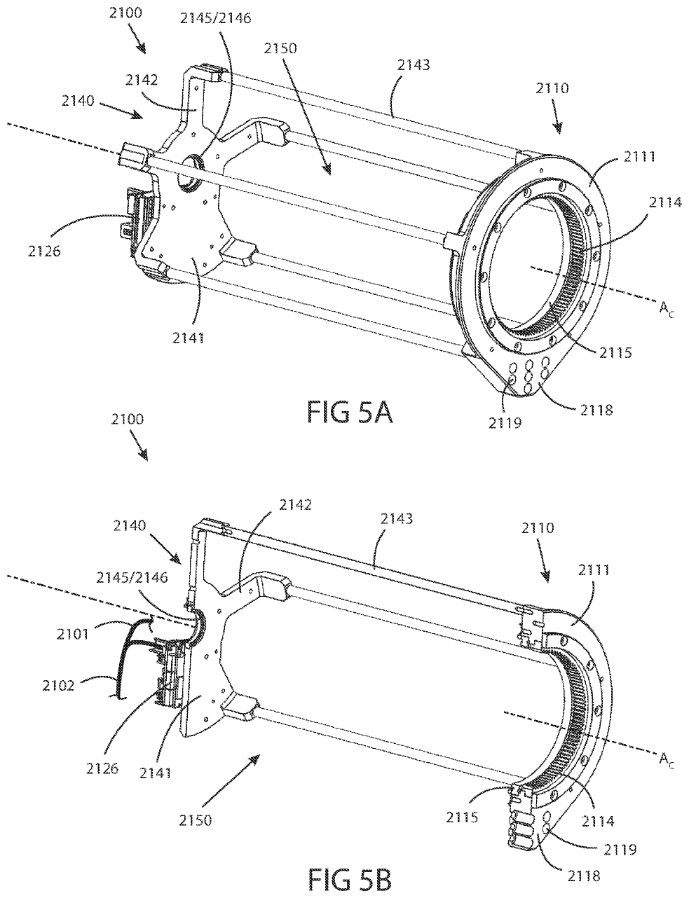

In some embodiments, the first ring 2211 includes a bearing surface 2213. The bearing surface 2213 can rotatably engage a corresponding bearing surface 2115 of the outer support assembly 2100, as described herebelow in reference to FIGS. 5A and B.

In some embodiments, the distal support assembly 2210 further includes a distal cover ring 2216. The distal support assembly 2210 can comprise one or more holes 2217, extending through the first ring 2211, the second ring 2212, and distal cover ring 2216. The embodiment of FIG. 4A includes eight holes 2217 that are positioned in a pattern around the circumference of the distal support assembly 2210. The distal support assembly 2210 can comprise a center hole 2218, surrounded by the first ring 2211, the second ring 2212, and distal cover ring 2216.

In some embodiments, the distal support assembly 2210 is fixedly attached to a first medial support ring, 2231, by one or more standoffs 2232.

Outer rotating assembly 2200 can comprise one or more motors 2225, such as ten motors 2225 shown in FIG. 4A. In some embodiments, outer rotating assembly 2200 comprises less than ten motors 2225. In other embodiments, outer rotating assembly 2200 comprises more than ten motors 2225. The distal ends of the motors 2225 can be fixedly attached to the distal support assembly 2210 and can be positioned between the distal support assembly 2210 and the first medial support ring 2231. Each motor 2225 can be attached to a rotatable gear, such as gears 2227, each positioned within a hole 2217, or gears 2228, 2229, each positioned within gap 2214. In the embodiment shown in FIG. 4A, gear 2228 is positioned to operably engage gear 2314 of the inner rotating assembly 2300 described hereabove in reference to FIG. 3. In some embodiments, gear 2228 rotates the inner rotating assembly 2300 with respect to the outer rotating assembly 2200 when driven by the attached motor 2225. In some embodiments, gear 2229 operably engages a mating gear 2114 of the outer support assembly 2100, as described herebelow in reference to FIGS. 5A and B, to rotate the outer rotating assembly 2200 relative to the outer support assembly 2100 when driven by the attached motor 2225.

In some embodiments, the first medial support ring 2231 is fixedly attached to a first support plate 2241, by one or more standoffs 2247.

In some embodiments, the first medial support ring 2231 and the first support plate 2241 are also attached with an inner and outer shell, 2233 and 2234, respectively, defining a space 2235 between the inner shell 2233 and the outer shell 2234.

In some embodiments, the inner shell 2233 and the orientation of motors 2225 define a volume, chamber 2250, within which the inner rotating assembly 2300 is rotatably positioned.

In some embodiments, the first support plate 2241 comprises a center hole 2242 including a bearing 2243 configured to rotatably receive the bearing surface 2343 of the inner rotating assembly 2300, as described hereabove in reference to FIGS. 3A and B. In some embodiments, the bearing 2315 of the inner rotating assembly 2300 is positioned between the first ring 2211 and the second ring 2212, and rotatably attached to the distal support assembly 2210. In some embodiments, the inner rotating assembly 2300 rotates relative to the outer rotating assembly 2200 riding on bearings 2243 and 2315, and is driven by motor 2225, rotating gear 2227, and opposing gear 2314.

In some embodiments, the outer rotating assembly 2200 comprises one or more motor controller assemblies 2226 that are operably attached to motors 2225. In some embodiments, the one or more motor controller assemblies 2226 provide power and/or control signals to motors 2225, and/or monitor the motors 2225. In some embodiments, the one or more motor controller assemblies 2226 are configured to monitor the current, the heat, and the rotational position of motors 2225 (e.g. when motor is servo) via one or more buses 2202.

In the embodiment shown in FIG. 4B, a first set of motor controller assemblies 2226 are fixedly attached to the proximal side of the first support plate 2241. In some embodiments, a second support plate 2244 is fixedly attached to the proximal ends of the first set of bearing surfaces 2246, and a second set of bearing surfaces 2246 is fixedly attached to the proximal side of the support plate 2244. In some embodiments, a proximal support plate 2245 is fixedly attached to the proximal ends of a second set of motor controller assemblies 2226. In some embodiments, a third shell 2249 surrounds the first and second sets of bearing surfaces 2246.

In some embodiments, the proximal support plate 2245 includes a bearing surface 2246 that rotatably engages a bearing surface 2146 of the outer support assembly 2100, as described herebelow in reference to FIGS. 5A and B.

In some embodiments, the proximal support plate 2245 supports a slip ring 2205 that rotatably attaches bus 2101 of the outer support assembly 2100 to bus 2201. Proximal portion 2206 of the slip ring 2205 rotates relative to distal portion 2207, which rotates with the outer rotating assembly 2200, while maintaining one or more electrical connections between bus 2101 and bus 2201. Bus 2201 daisy chains to each motor control assembly 2226, which each operably attach to motors 2225.

The motor control assemblies 2226 along with the first support plate 2241, support plate 2244, and the proximal support plate 2245 define a volume, chamber 2355, within which buses 2201 (e.g. wires) are routed and connect to the motor control assemblies 2226, such as via connectors 2203. One or more bus 2202, between the motor controller assembly 2226 and motors 2225, are routed within space 2235, outside of the chamber 2250.

Referring to FIG. 5A, a perspective view of the outer support assembly 2100 is illustrated, in accordance with embodiments of the present inventive concepts. Referring additionally to FIG. 5B, a perspective cross-sectional view of the outer support assembly 2100 is illustrated, in accordance with embodiments of the present inventive concepts.

In some embodiments, the outer support assembly 2100 comprises a cylindrical structure, surrounding a volume 2150, within which the outer rotating assembly 2200 and inner rotating assembly 2300 are rotatably positioned.

In some embodiments, the outer support assembly 2100 extends along the central axis A_{C}, but does not rotate about the central axis A_{C}.

In some embodiments, the outer support assembly 2100 comprises distal support assembly 2110. The distal support assembly 2110 can comprise a first ring 2111, operably attached to a bearing 2115. Bearing 2115 rotatably engages a bearing surface 2213 of outer rotating assembly 2200, as described hereabove in reference to FIGS. 4A and B.

The distal support assembly 2110 can further include a ring gear, gear 2114, which operably engages a mating gear of 2200 (e.g. gear 2229), to rotate the outer rotating assembly 2200 relative to the outer support assembly 2100 when driven by a motor 2225 of the outer rotating assembly 2200.

In some embodiments, the distal support assembly 2110 includes a base portion 2118. The base portion 2118 can include one or more mounting holes 2119 for attaching to a translation assembly 2400, as described herein.

In some embodiments, the outer support assembly 2100 includes a proximal support assembly 2140, comprising plate 2141 and struts 2142. In some embodiments, the distal support assembly 2110 is fixedly attached to the proximal support assembly 2140 via one or more standoffs 2143, such as five standoffs 2143 shown in FIG. 5A. This structure defines a volume 2150.

In some embodiments, the plate 2141 comprises a center hole 2145 with a bearing 2146, configured to rotatably receive the bearing surface 2246 of the outer rotating assembly 2200. The bearing surface 2213 of the outer rotating assembly 2200 can be positioned within and rotatably attached to the bearing 2115. In some embodiments, the outer rotating assembly 2200 rotates relative to the outer support assembly 2100 riding on bearings 2115 (e.g. distally) and bearing 2146 (e.g. proximally), and is driven by a motor 2225, rotating on gear 2229, and opposing gear 2114.

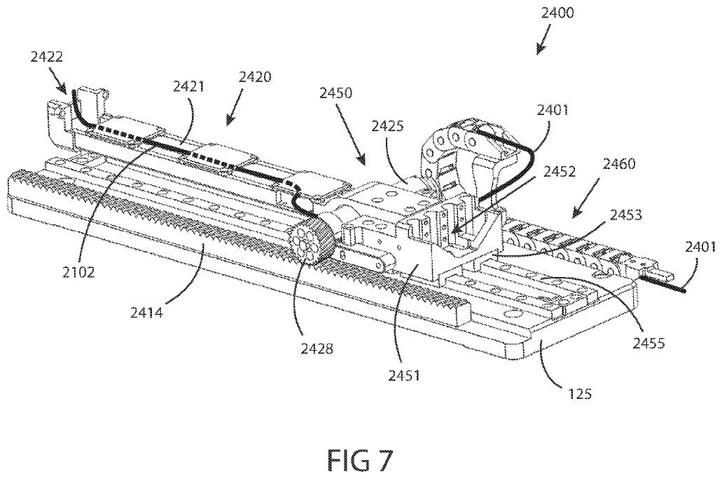

In some embodiments, the outer support assembly 2100 comprises one or more motor controller assemblies 2126, such as one motor controller assembly 2126 shown in FIG. 5B. The motor controller assembly 2126 can operably attach to a motor 2425 of the linear drive assembly 2400, as described herebelow in reference to FIG. 7. The motor controller assembly 2126 can provide power and control signals to motor 2425, and/or monitor motor 2425. In some embodiments, the motor controller assembly 2126 is configured to monitor the current, heat, and rotational position of motor 2425 (e.g. when motor is servo) via one or more busses 2102.

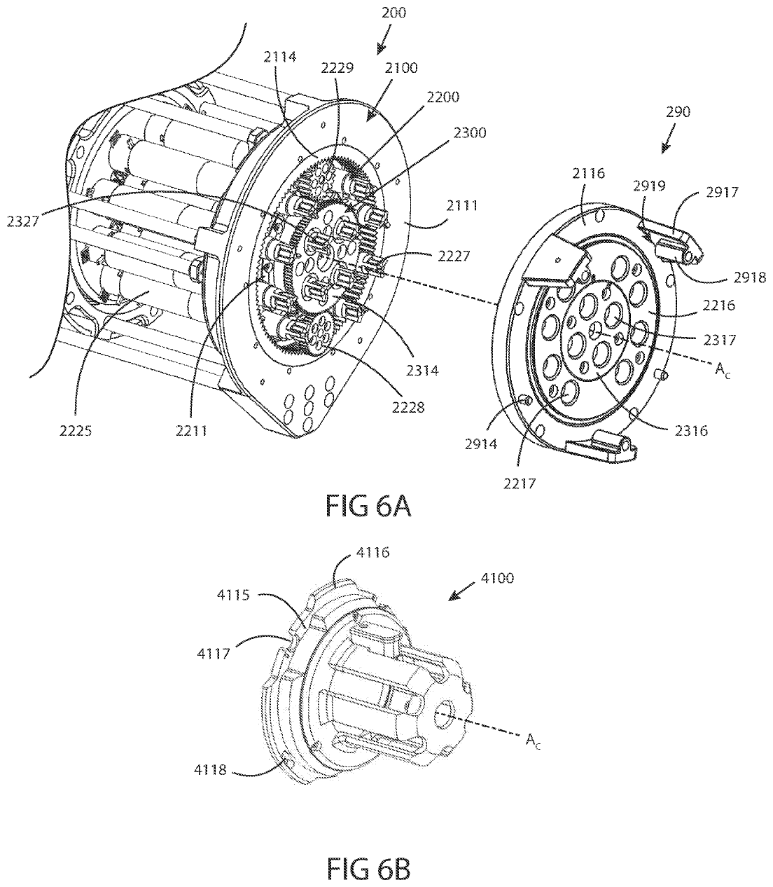

Referring to FIG. 6A, a perspective view of the outer support assembly 2100, the outer rotating assembly 2200, and the inner rotating assembly 2300 concentrically positioned to form the tool drive 200 along with an interface assembly 290 is illustrated, in accordance with embodiments of the present inventive concepts.

In some embodiments, gear 2229 engages mating gear 2114. In some embodiments, the motor 2225 rotates gear 2229 to cause a gear 2229 to rotate relative to the outer support assembly 2100.

In some embodiments, gear 2314 engages gear 2228, and the motor 2225 rotates gear 2228 to cause the inner rotating assembly 2300 to rotate relative to the outer rotating assembly 2200. In some embodiments, the inner rotating assembly 2300 is driven to rotate in the clockwise direction or counter clockwise direction while the outer rotating assembly 2200 is fixed or otherwise not presently rotating. In some embodiments, the inner rotating assembly 2300 is driven to rotate in a clockwise or counter clockwise direction that is a different direction of rotation than the present direction of rotation of the outer rotating assembly 2200. In some embodiments, the inner rotating assembly 2300 is driven to rotate in a clockwise or counter clockwise direction that is a same direction of rotation and a same angular velocity as that of the present direction of rotation and angular velocity of the outer rotating assembly 2200 so that the inner rotating assembly 2300 and outer rotating assembly rotate together in unison. In some embodiments, the inner rotating assembly 2300 is driven to rotate in a clockwise or counter clockwise direction that is a same direction of rotation as that of the present direction of rotation of the outer rotating assembly 2200, but at a different angular velocity than that of the outer rotating assembly 2200.

An interface assembly, interface 290, is shown removed from the tool drive 200. In some embodiments, the interface assembly 290 is removable, and sterilizable and/or replaceable to form a sterile interface between a tool 400 and the tool drive 200.

In some embodiments, the interface assembly 290 includes plate 2316 of the inner rotating assembly 2300, the ring 2216 of the outer rotating assembly 2200, and an outer ring 2116, each rotatable relative to each other about axis A_{C}. In some embodiments, the plate 2316 removably attaches to gear 2314 of the inner rotating assembly 2300. In some embodiments, the ring 2216 removably attaches to the first ring 2211 of the outer rotating assembly 2200, and the ring 2116 removably attaches to the first ring 2111 of the outer support assembly 2100, such that each component including the plate 2316, the ring 2216, and the outer ring 2116, rotates with gear 2314, the first ring 2211, and the first ring 2111, respectively.

Each gear 2327, 2227 can align with a hole 2317, 2217, respectively, of the plate 2316 and the ring 2216, respectively.

In some embodiments, the outer ring 2116 includes one or more projections 2917 extending parallel to axis A_{C}. In some embodiments, each projection 2917 comprises an inward radial projection 2918, defining a space 2919, between the proximal end of the inward radial projection 2918 and ring 2116. The outer ring 2116 can include one or more alignment pins 2914.

Referring additionally to FIG. 6B, a perspective view of an interface assembly 4100 for connecting a tool 400 to the interface assembly 290 is illustrated, in accordance with embodiments of the present inventive concepts. The tool 400 includes an interface assembly 4100, comprising a proximal ring 4115.

In some embodiments, the proximal ring 4115 includes one or more radial projections 4116, and one or more recesses 4117, and one or more alignment channels 4118. The proximal ring 4115 can be aligned with the interface assembly 290, such that projection 2918 aligns with recess 4117, and interface assembly 4100 is moved proximally along central axis A_{C} to mate with interface assembly 290. One or more components of tool 400 can engage with the outer rotating assembly 2200 and/or the inner rotating assembly 2300, and the interface assembly 4100 can be rotated (while at least a portion of the tool assembly 400 does not rotate, such as the portions configured to engage with assemblies 2200, 2300). Projection 4116 can rotate towards and into the space 2919, removably attaching interface assembly 4100 to interface assembly 290 and/or tool drive 200. Alignment pins 2914 can slidingly and/or frictionally engage alignment channels 4118. Tool 440 can be of a similar constructed and arrangement as described in applicant's co-pending application U.S. Provisional Application No. 62/614,225, filed Jan. 5, 2018, the content of which is incorporated herein by reference.

Referring to FIG. 7, a perspective view of a linear drive assembly 2400 is illustrated, in accordance with embodiments of the present inventive concepts. The linear drive assembly 2400 includes a carriage 2450 and a base 2451.

In some embodiments, the base 2451 includes a mating portion, a recess 2452 for fixedly attaching to base 2118 (e.g. via screws through the one or more mounting holes 2119) of the outer support assembly 2100.

In some embodiments, the carriage 2450 includes motor 2425 operably attached to gear 2428. In some embodiments, the base 2451 secures the motor 2425 such that the gear 2428 aligns with a linear rack gear 2414, fixedly attached to a frame, such as carriage 125 as described hereabove in reference to FIG. 1.

In some embodiments, the carriage 2450 includes one or more linear bearings 2453, slidingly engaged with one or more linear rails 2455 also fixedly attached to the carriage 125, such that the carriage 2450 translates along the linear rails 2455 when driven by the motor 2425.

In some embodiments, a support arm 2420 extends proximally from the carriage 2450. In some embodiments, the support arm 2420 includes a channel 2421 through which buses 2102 can be routed. A connector 2422 is positioned at the proximal end of support arm 2420 and can be configured to connect to plate 2141 and/or controller assembly 2126, as described hereabove in reference to FIGS. 5A and B. Bus 2102 is routed from the motor controller assembly 2126 to the motor 2425. The support arm 2420 provides support for the proximal portion of the outer support assembly 2100.

The linear drive assembly 2400 includes a cable management assembly 2460, surrounding a bus 2401, operably attached to the motor 2425 and/or other components of the tool drive 200.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. A system for performing a medical procedure on a patient, comprising:
an articulating probe assembly, comprising:
an inner probe comprising multiple articulating inner links;
an outer probe surrounding the inner probe and comprising multiple articulating outer links; and
at least two working channels that exit a distal portion of the probe assembly,
at least one tool configured to translate through one of the at least two working channels; and
a manipulator for controlling the at least one tool.

2.-12. (canceled)
